(19) United States
(12) Patent Application Publication
(10) Pub. No.: US 2009/0076388 A1
(43) Pub. Date: Mar. 19, 2009

(54) LINEAR WAVE INVERSION AND DETECTION OF HARD OBJECTS

(75) Inventors: Joshua Napoli, Arlington, MA (US); Sandy Stutsman, Watertown, MA (US)

Correspondence Address: CANTOR COLBURN, LLP, 20 Church Street, 22nd Floor, Hartford, CT, 06103, US

(73) Assignee: ACTUALITY SYSTEMS, INC., Bedford, MA (US)

(21) Appl. No.: 12/212,919

(22) Filed: Sep. 18, 2008

Related U.S. Application Data
(60) Provisional application No. 60/973,306, filed on Sep. 18, 2007.

(51) Int. Cl. A61B 8/00

(52) U.S. Cl. 600/437

(57) ABSTRACT

A method for providing an ultrasound image, includes: assembling data from ultrasound image data of a subject; performing linear wave inversion to provide a reflectivity volume from the assembled data; and performing vision processing to identify and localize features within the ultrasound image.

### PRIORITY CLAIM AND RELATED APPLICATIONS

This application claims the benefit under 35 U.S.C. §119(b) of U.S. Provisional Patent Application Ser. No. 60/973306, filed Sep. 18, 2007 and entitled “Ultrasound Linear Wave Inversion and Detection of Hard Objects,” which is hereby incorporated by reference in its entirety.

### BACKGROUND OF THE INVENTION

1. Field of the Invention

The teachings herein relate to ultrasound imaging and particularly to ultrasound imaging of hard surfaces within tissue.

2. Description of the Related Art

In medical imaging, ultrasound pulses are conducted into the body and the timing and intensity of echoes are plotted to produce an image of the tissues through which the pulses traveled. A major problem with medical ultrasound imaging is that the ultrasound pulse becomes distorted as it propagates through the body. One source of the distorting may come from attenuation.

Attenuation and other dispersion effects cause the ultrasound pulse to spread out. Attenuation in tissue is frequency-dependent and higher frequencies are attenuated at a greater rate than lower frequencies. When a strong reflector, such as a prostate brachytherapy seed, is imaged, the spread of the ultrasound pulse is visible as a bright tail, extending away from the ultrasound pulse origination location (ultrasound transducer). Weak reflections have a proportionally weaker tail that normally become a component of the noise in deeper parts of an image.

The distortions described above are particularly detrimental when attempting to identify and localize hard objects embedded in tissue. Consider, for example, use of ultrasound in brachytherapy treatment. In prostate brachytherapy, typically 100 small (4.5 mm long, 0.8 mm diameter) radioactive seeds need to be precisely positioned within the prostate according to a dose prescription and associated “pre-plan.” During the implant operation, ultrasound is used to image the positions of needles and seeds. Since these are hard, metal objects, distortion of the ultrasound pulse produces bright artifacts in a typical brightness-mode (B-mode) display. These artifacts make it difficult to identify seeds and obscure the true positions of needles and seeds making it more difficult to follow the pre-plan and arrange the seeds in the desired locations.

A variety of techniques for improvement of image quality from such ultrasound imaging have been attempted. Some of these are now presented and discussed for perspective.

Ultrasound Computer Tomography (USCT) provides one method for dealing with this problem. In USCT, attenuation at every point within a volume or plane is calculated, based on measurements from various directions. Since measurements are taken from all directions, direction-dependent artifacts in the underlying signal collection do not necessarily result in localized artifacts in the tomographic reconstruction. Unfortunately, USCT is generally impractical for medical use because transducers must surround the imaged object in a water bath. This configuration means that only externally-accessible organs may be imaged, such as the breast. Furthermore, a large number of transducers and data acquisition channels are used, making the equipment prohibitively expensive.

Synthetic Aperture Ultrasound is another technique where information for a point is gathered from a range of directions. Synthetic Aperture Ultrasound is most effective when a large transducer area is used. Small transducers for urological and surgical applications may not see a substantial benefit.

Vibro-acoustography is an imaging approach where the resonant properties of each point in the imaged volume are measured. Frequency-dependent attenuation does not corrupt the image because the frequency difference between two pulses excites the imaged point. Vibro-acoustography is too slow for real-time medical imaging. In B-mode ultrasound, one echo round-trip collects data for all points along one or more beams, but in vibro-acoustography, the same period of time is used to collect data for just one point. Furthermore, vibro-acoustography also uses specialized probes and signal processing, presenting certain maintenance and expense requirements. “Vibro-Acoustography” is an approach using two custom ultrasound probes with intersecting beams and different frequencies. These are used to both excite and image points within the target volume. The frequency difference between the probes is set to the natural resonant frequency of the seeds. Resonating seeds can be readily distinguished from prostate tissue. However this technique has not been tested under realistic conditions and it is not obvious whether practical control circuitry and probes could be developed to scan a prostate in real-time.

A more practical technique for overcoming ultrasound pulse spreading would use conventional transducers and scanners while inverting the spreading though signal processing. In “impediography”, deconvolution by an incident wave was used to derive the impulse response of small samples. More recently, spiking deconvolution and blind deconvolution have been applied to so-called A-scans for USCT to account for the transducer transfer function. The deconvolution approach assumes that one function has been convolved with the entire A-scan. As noted above, the incident and reflected ultrasound wave is distorted due to dispersion and therefore deconvolution by an ideal incident wave is not sufficient to recover the material impulse response.

So far, no automatic prostate brachytherapy seed detection algorithm has been proven to be reliable in human data. Most researchers have focused on fusing fluoroscopic imaging with ultrasound. Metal seeds show up with high contrast in fluoroscopic imaging. It is possible to overcome the problem of overlapping seeds in the projection when multiple image directions are used. Although effective for seed imaging, fluoroscopic imaging will always be highly inconvenient during permanent prostate implant operations. The equipment is cumbersome, and radiation safety is troublesome to maintain for the staff in the room. Obtaining multiple view directions is often impractical, due to space constraints and the lithotomy position of the patient.

Most previous attempts at ultrasound seed detection have been based on B-mode imagery. Not even expert physicians are able to accurately search for seeds within a B-mode ultrasound scan. The contrast in the image is simply not enough. It is difficult to distinguish between blood, needles, seeds, and calcifications in B-mode images. The evidence indicates that neither person nor machine can reliably find seeds in “static” B-mode imagery, and that improving B-mode image quality is not the correct approach. Instead, the seed finder must incorporate some other type of information not available from a static ultrasound volume.

Signature Methods attempt to find seeds by searching for seed-like patterns in the B-mode imagery. The first attempt at ultrasound seed detection used “CFAR processors” to detect similar sub-images of the ultrasound slice. It had comparatively good results, but was slow and would not work for angulated seeds. A Trans-Urethral Ultrasound Probe was implemented at the Mayo Clinic, and promises better image quality, but so far, has failed to improve the automatic detection problem.

Singular Spectrum Analysis has been used to detect the repetitive tail that often follows seeds in B-mode ultrasound. The tail is dependant on the angles between the ultrasound beam and the seed, and therefore the technique does not work with angulated seeds. Better modeling of the cause of the tail would be required in order to generalize this approach. If the imaging plane is not well aligned with the seed, then the B-mode image may not show a visible tail.

The structure of the implant operation can be used to help the seed finder. When the needle is withdrawn, the search for seeds can then be restricted to a cylinder around the needle path. Unfortunately, the needle detection implies many 3-D scans need to be done. Oncologists who are used to a classical technique will resist doing this extra work. Further tests on human data are scant and have not been published.

Doppler ultrasound can be used to detect minute movements within the body (a fraction of a wavelength). Scanners and TRUS probes used in urology operating rooms are capable of Doppler imaging. Implanted seeds are small and sit in an elastic medium and can actually be excited by the ultrasound signal itself Typically, a 1 MHz power Doppler imaging system will detect the seeds. Unfortunately, the seeds can not be detected at any higher frequency resonance.

“Ultrasound-Elastography” uses raw ultrasound before and after a slight compression of tissue. Implanted seeds are much harder than the surrounding medium. If the prostate is compressed, then Doppler imaging could be used to highlight the hard seeds. A larger driving force is required than can be delivered by the ultrasound probe. Unfortunately, this means that the method requires additional devices to deliver the required energy. A magnetic field has been used to vibrate a seed loaded with a magnetizable component. This technique is not practical because it renders the patient ineligible for MRI and could destroy the ultrasound probe by inducing a current in it.

Researchers have tried Vibro-Elastography to image the prostate and implanted seeds. This technique involves vibrating the ultrasound probe. A correlation function (speckle tracking) is run between successive RP ultrasound frames. Speckle tracking was found to fail in the face of the shadows cast by seeds and needles within a phantom. In humans, bleeding and trauma would cause additional shadows, probably rendering this approach useless.

Thus, techniques for improving the quality of ultrasound images are needed. Preferably, the techniques provide an ultrasound-only seed detection technology for hard object identification and localization. Preferably, the technique provides improvement of the quality of all ultrasound images, and in particular, to images such as those obtained during brachytherapy.

### BRIEF SUMMARY OF THE INVENTION

In an embodiment, a method for providing ultrasound image data, is provided and includes: assembling data from ultrasound image data of a subject; performing linear wave inversion to provide a reflectivity volume from the assembled data; and performing vision processing to identify and localize hard objects within the ultrasound image.

One embodiment of the present invention is directed to a method for providing an ultrasound image data. The method of this embodiment includes assembling data from ultrasound image data of a subject; performing linear wave inversion on the assembled data to provide a reflectivity image; performing vision processing on the reflectivity image to identify and localize features within the ultrasound image; and displaying the ultrasound image including the features.

Another embodiment of the present invention is directed to a system for identifying hard objects. The system of this embodiment includes an ultrasound scanner that creates an RF image representing an imaged area, a signal processor that converts the RF image to a reflectivity image by performing linear wave inversion and a computer vision processor coupled to the signal processor to locate hard objects in the reflectivity image.

Another embodiment of the present invention is directed to a method for determining locations of one or more element of interest in a subject. The method of this embodiment includes assembling data from ultrasound image data of the subject; performing linear wave inversion on the assembled data to provide a reflectivity image; and analyzing the reflectivity image to determine the location of elements of interest.

Another embodiment of the present invention is directed to a computer program product for determining locations of one or more element of interest in a subject. The computer program product of this embodiment includes a storage medium readable by a processing circuit and storing instructions for execution by the processing circuit for facilitating a method including: assembling data from ultrasound image data of the subject; performing linear wave inversion on the assembled data to provide a reflectivity image; and analyzing the reflectivity image to determine the location of elements of interest.

### BRIEF DESCRIPTION OF THE FIGURES

The subject matter which is regarded as the invention is particularly pointed out and distinctly claimed in the claims at the conclusion of the specification. The foregoing and other features and advantages of the invention are apparent from the following detailed description taken in conjunction with the accompanying drawings in which:

FIG. 1 is a flowchart of the data processing pipeline of the system;

FIG. 2 shows a diagram of a transducer coupled to an Imaged Object through a medium and includes a pressure wave has been imparted to the Imaged Object;

FIG. 3 shows a diagram of a transducer coupled to an Imaged Object as the transducer receives a backscatter pressure wave;

FIG. 4 is a diagram of a reflectivity basis utilized according to one embodiment of the present invention that includes envelopes of several basis elements graphed and corresponding to reflectors at different distances from the transducer, whose RF signals are plotted at differing time steps.

FIGS. 5a and 5b, show two different types of RF images with FIG. 5a showing a diagram of two RF signal pre-images and FIG. 5b showing a diagram highlighting the overlap of two RF signal pre-images;

FIG. 6 is a B-mode ultrasound image of a seeded human prostate with seed locations as determined according to an embodiment of the present invention;

FIG. 7 is a flowchart of the generation of the Reflective Power Volume from an input Reflectivity Image and includes power averaging, median filtering and thresholding;

FIG. 8 is a flowchart that takes the Classification Volume through Blob Analysis producing detected seed locations; and

FIG. 9 is a flowchart of the Principal Component Analysis used as part of the Blob Analysis; and

FIG. 10 is a MATLAB (The MathWorks, Inc.) source listing for calculating a homogeneous transform matrix corresponding to an inertia tensor.

### DETAILED DESCRIPTION OF THE INVENTION

Disclosed is a technique for a deconvolution process for ultrasound images. The technique is referred to as “Linear Wave Inversion” (LWI). Unlike the prior art, LWI is sensitive to objects having a dense reflectivity basis. The resulting signal is a measurement of the reflectivity of the imaged object. LWI is especially useful in combination with a computer vision system designed to detect hard objects. By clarifying the ultrasound signature of hard objects, LWI simplifies the problem of recognizing hard objects. This is accomplished by, among other things, use of automatic computer vision techniques. A computer vision system that works in conjunction with LWI is also provided herein.

The following description is directed generally to LWI in the context of brachytherapy treatment. Of course, the teachings are not limited to this context unless specifically noted. As noted, the teachings herein are illustrative only and the scope of the present invention is limited only by the appended claims.

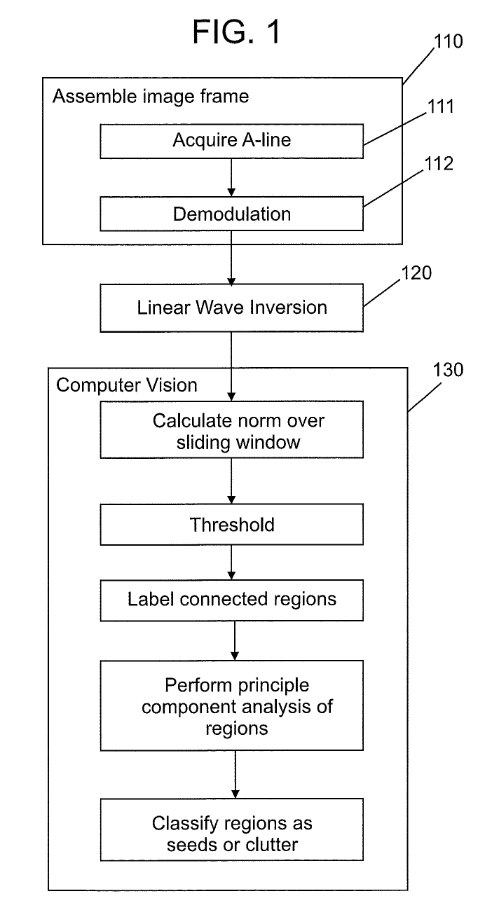

Referring now to FIG. 1, aspects of an embodiment of the present invention are shown in a data flow diagram. First, an ultrasound image frame is assembled at a block 110. Signal processing is performed at a block 120 to convert from raw echo-data to an inference about what impedances caused those echoes, or in terminology used herein, conversion from an RF image to a reflectivity image is performed. Conversion, as discussed herein, is also referred to as “linear wave inversion.” Generally, linear wave inversion is the process of inferring a plurality of reflector amplitudes that most closely matches an observed RF image. The reflector amplitudes are typically denoted in a vector.

At a block 130, computer vision techniques are applied to the reflectivity image created at block 120 to extract hard features. In one embodiment, the computer vision techniques and the image assembly of block 110 work together synergistically to provide for hard object detection. However, it should be understood that such aspects may also be performed separately or in conjunction with other techniques. For example, the reflectivity image could be used to make an image of the tissue with fewer artifacts than a B-mode image, or the computer vision techniques could be applied directly to B-mode imagery to detect objects with strong echoes.

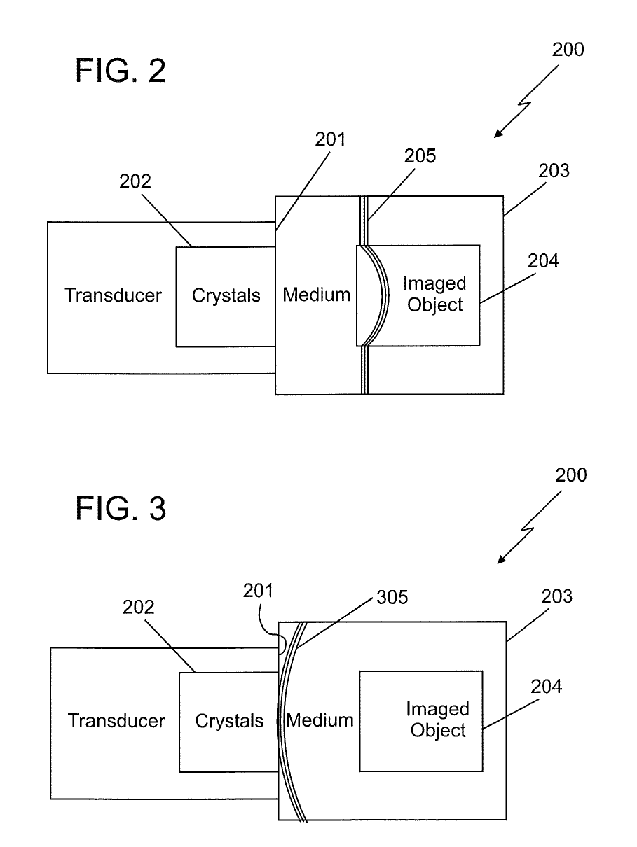

Referring now to FIG. 2, it will be understood that the Linear Wave Inversion (LWI) taught herein is applicable to ultrasound backscatter or attenuation signals from an ultrasound system 200. Ultrasound signals are generally acquired using one or more crystals 202 coupled through a medium 203 to an imaged object 204. The crystals 202, in one embodiment, may be electrically excited to impart energy in the forms of pressure waves 205 through the medium 203 to the imaged object 204.

FIG. 3 shows the system 200 shown in FIG. 2 including reflected pressure waves 305. Within a short period of time after the pressure waves 205 (FIG. 2) are created, the energy is dissipated. Much of the energy is converted into heat within the imaged object, but some of the energy leaves the imaged object 204 as reflected pressure waves 305 and can be measured according to methods known in the prior art.

Medical ultrasound imaging is generally concerned with backscatter that intersects the same ultrasound probe that imparted the energy. In ultrasound computed tomography and industrial applications, the crystals 202 may include transmitting crystals and receiving crystals that may be in separate locations. Pressure waves 305 that exit the imaged object 204 and intersect the receiving crystals (202) induce electrical signals in the crystals.

Referring back to FIG. 1, the ultrasound scanner amplifies the electrical signals (or “Acquire A-line”) from the receiving crystals at a block 111. The amplified signal is typically demodulated and digitized at a block 112. The digitized, demodulated signal is typically called the “RF signal”. Typically, the ultrasound system has been arranged such that the RF signal from one excitation corresponds to features within the imaged object near a particular geometric ray that passed through the imaged object. Of course, synthetic aperture ultrasound provides an exception: each measurement of the imaged object generally corresponds to an extended area or volume of the object and further processing is required to form a traditional pixilated characterization of the imaged object.

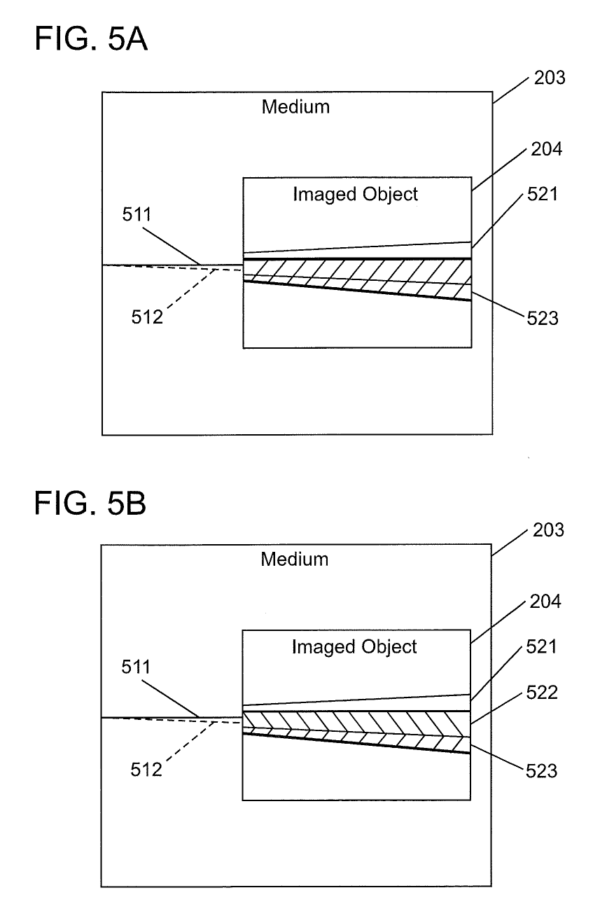

Referring now to FIGS. 5a and 5b, examples of RF signal components are shown. The “RF signal pre-image” 521 is the region of the imaged object 204 whose qualities influence the RF signal. The “beam direction” is the direction of the central ray 511 from the transducer through the RF signal pre-image 521. Excitations and measurements can be performed along other beam directions (using phased array steering, for example).

A second beam direction 512 and RF signal pre-image 523 are shown in FIG. 5b. In FIG. 5b, the overlap of the pre-images 521 and 523 is drawn as a dense stippled area 522. In medical imaging, a set of beam directions is used to acquire information about a 2-D slice or 3-D wedge of the body. The “RF image” is the set of RF signals corresponding to the set of beam directions. RF signals and RF images are plots of pressure wave's 305 (FIG. 3) amplitude and phase over elapsed time since excitation.

As discussed above, one embodiment of the present invention utilizes Linear Wave Inversion to resolve or clarify ultrasound images. In particular, Linear Wave Inversion may be utilized to process an “RF image.”

In LWI, a “reflectivity image” is a plot of the fraction of incident acoustic pressure that is reflected towards the transducer at every point of the imaged object. Linear Wave Inversion is the process of computing a reflectivity image that most closely matches the observed RF image.

In LWI, the reflectivity image is modeled as a linear combination of basis elements. In the simplest case, a basis element corresponds to an RF signal along a particular beam direction. The “continuous reflectivity basis” is the set of all basis elements for an imaged object. For example, a reflectivity basis may express the RF image of a feature, such as a brachytherapy seed in a medium, at various distances along the beam axis. A reflectivity basis may alternatively include information about the RF image due to a plurality of feature-types. These features may also include angulation, surface roughness, mass, or acoustic impedance.

In practical calculations, a “discrete reflectivity basis” must be used instead of a “continuous reflectivity basis.” The discrete reflectivity basis is a discrete sampling of the continuous reflectivity basis along the distance characterization parameter. That is, to sample a continuous reflectivity basis to derive a discrete reflectivity basis, combine a low-pass filter (to satisfy the sampling theorem) with the continuous reflectivity basis and evaluate it at the discrete sample steps. A practical discrete reflectivity basis would use one basis sample per central frequency wavelength, for example 250 microns. A typical reflectivity basis used with medical backscatter ultrasound would have a 6 MHz central frequency and a 10 cm deep region of interest. The speed of sound in tissue is roughly 1500 m/s. Therefore, a typical discrete reflectivity basis has 400 elements (=10 cm/250 microns).

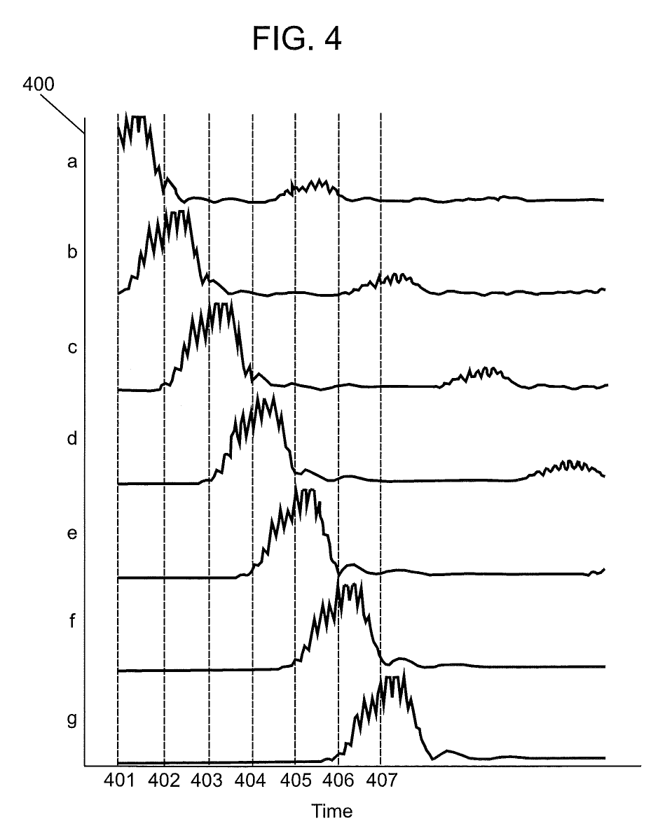

FIG. 4 shows a graph of that characterizes the impact on the RF image of a single thin reflector in a plurality of different particular positions a-g. In one embodiment, the information may be created by tests done in water. The different positions a-g each include particular times when the reflected pressure wave was received. In one embodiment, the graph shown in FIG. 4 could be considered, for example, as the basis for a look up table. For example, the RF signal for a portion of an imaged object with reflecting features at time 404 and 407 with amplitudes 0.1 and 0.2 would be the sum of 0.1 times the basis element d and 0.2 times the basis element g.

Referring again to FIGS. 5a and 5b, a basis element preferably characterizes the entire RF signal pre-image 521 for a beam direction 511. In general, the RF signal pre-images (521 and 523) for nearby beam directions (511 and 512) overlap in an area 522. When the reflectivity basis element characterizes the overlapping RF signal pre-images 521 and 523, the best use of the overlapping measurements is made, resulting in the reflector image being properly sharpened in the lateral direction. Typically, the reflectivity basis is identical along every beam direction. Therefore, basis elements may only need to be stored for one beam direction. The basis elements for a given beam direction can be constructed using the transformation between the stored beam direction and the given beam direction.

The discrete reflectivity basis can be represented as a matrix B, where each basis element forms a column. Then, for reflectivity image column-vector u and RF image column-vector v, the linear model equates the RF image column-vector to the matrix product of the discrete reflectivity basis and the reflector column vector plus noise e: B u+e=v. Given B and v, and assuming e is irreducible in terms of B, we can estimate u in the least squared error sense. With a typical discrete reflectivity basis of 400 elements, it is feasible to use singular value decomposition, a standard technique, to estimate u Examples of software that performs singular value decomposition are: the GNU Scientific Library, MATLAB, and Mathematica.

In one embodiment, the reflectivity basis B maps a reflection vector u to RF measurement v (Bu=v). The reflection vector u corresponds to at least one row of the RF image (that is, along the beam axis). The ultrasound transducer typically does not have a very sharp point spread function. Therefore, the elements of a particular RF image row are affected by reflectors that are nearby but not directly on the axis of the row. In this case, it is beneficial for the reflectivity basis B to relate a reflection vector to multiple rows of the RF image. Below an illustration of the case where B relates a reflection vector to three RF image rows is shown. Elements of the reflectivity basis B_{x,δ,t} give the contribution of the reflector at distance x along beam axis I (u_{i,x}) to the RF intensity measured for beam j at time t (v_{i,t}). That is, the information from the graph shown in FIG. 4 may be described in the reflection basis B. Ultimately, solving for the reflection vector u results in a series of values that may be used by the computer vision described below to show an image including the hard objects.

$$( \begin{matrix}{B}_{1 , - 1 , 1} & {B}_{1 , - 1 , 2} & … & {B}_{1 , - 1 , N} \\ {B}_{2 , - 1 , 1} & {B}_{2 , - 1 , 2} & … & {B}_{2 , - 1 , N} \\ & ⋮ & & \\ {B}_{M , - 1 , 1} & {B}_{m , - 1 , 2} & … & {B}_{M , - 1 , N} \\ {B}_{1 , 0 , 1} & {B}_{1 , 0 , 2} & … & {B}_{1 , 0 , N} \\ {B}_{2 , 0 , 1} & {B}_{2 , 0 , 2} & … & {B}_{2 , 0 , N} \\ & ⋮ & & \\ {B}_{M , 0 , 1} & {B}_{M , 0 , 2} & … & {B}_{M , 0 , N} \\ {B}_{1 , 1 , 1} & {B}_{1 , 1 , 2} & … & {B}_{1 , 1 , N} \\ {B}_{2 , 1 , 1} & {B}_{2 , 1 , 2} & … & {B}_{2 , 1 , N} \\ & ⋮ & & \\ {B}_{M , 1 , 1} & {B}_{M , 1 , 2} & … & {B}_{M , 1 , N}\end{matrix} ) \cdot ( \begin{matrix}{u}_{i , 1} \\ {u}_{i , 2} \\ ⋮ \\ {u}_{i , N}\end{matrix} ) = ( \begin{matrix}{v}_{i - 1 , 1} \\ {v}_{i - 1 , 2} \\ ⋮ \\ {v}_{i - 1 , M} \\ {v}_{i , 1} \\ {v}_{i , 2} \\ ⋮ \\ {v}_{i , M} \\ {v}_{i + 1 , 1} \\ {v}_{i + 1 , 2} \\ ⋮ \\ {v}_{i + 1 , M}\end{matrix} )$$

Linear wave inversion has been described in terms of backscatter (reflectivity) imaging typically used in medical ultrasound imaging. It should be apparent that the linear wave inversion is also applicable in absorption and backscatter varieties of ultrasound computed tomography.

The ultrasound probe can be rotated or translated while collecting an array of RF images. The rotation or translation can be accomplished by any of a variety of means, including by hand, by a servo attached to the crystals or to the transducer or by phased array beam steering. An array of RF images that are suitably spaced and referenced to the transducer orientations can be thought of as an “RF volume”. An RF volume can be converted into a corresponding reflectivity volume by performing linear wave inversion on each RF image. An improved reflectivity volume can be achieved by using basis elements that characterize 3-D RF signal pre-images. In this case, a single linear wave inversion is performed and the reflectivity volume is properly sharpened in both directions that are orthogonal to the beam directions. In the same manner, very rapid scanning or gated scanning can be used to build up a 4-D array of RF images and reflectivity images.

In one embodiment, the reflectivity volume is made up of x×y×N samples. Typically, assuming a 20 MHz sampling rate, a 10 cm-deep imaged object, and a speed of sound of 1,500 m/s, y=1,333 samples. x is a function of the imaging characteristics of the transducer crystals and is approximately 200 samples. In instances where volume-scanning is performed, 10≦N≦100.

In short, linear wave inversion of an RF signal results in a reflectivity volume which may be processed by computer vision techniques for hard object identification and localization. Consider now aspects of computer vision for hard object identification and localization. Of course, in embodiments of the present invention may include processing in addition to or the place of computer vision. For example, the reflectivity volume (or reflectivity image if the information is in less than three dimensions) may be processed by programs that perform dosimetry calculations

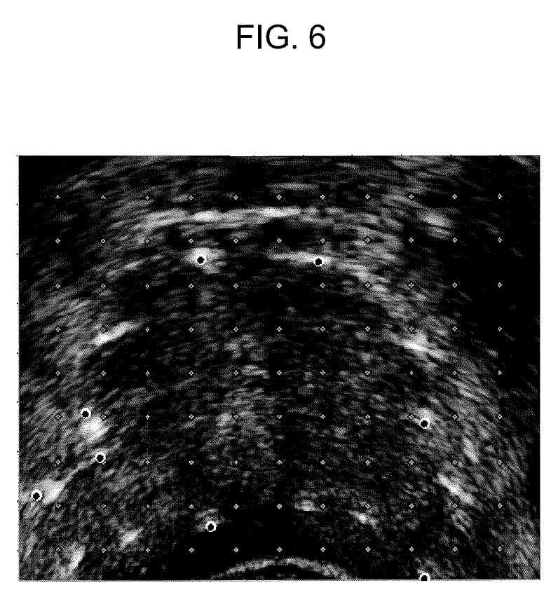

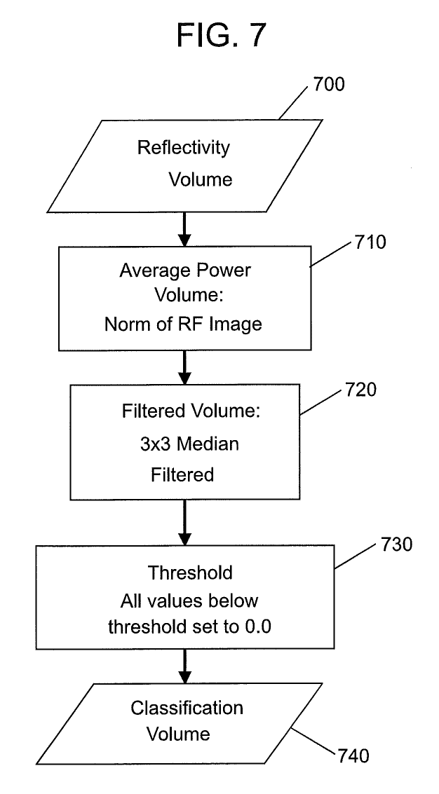

Aspects of computer vision for hard object identification and localization are depicted with regards to FIGS. 7 through 9. That is, FIGS. 7 through 9 describe the conversion of the reflectivity volume into a collection of ellipsoidal features that correspond to hard objects in the imaged object. The results can be illustrated overlaid on a standard B-mode image, as shown in FIG. 6.

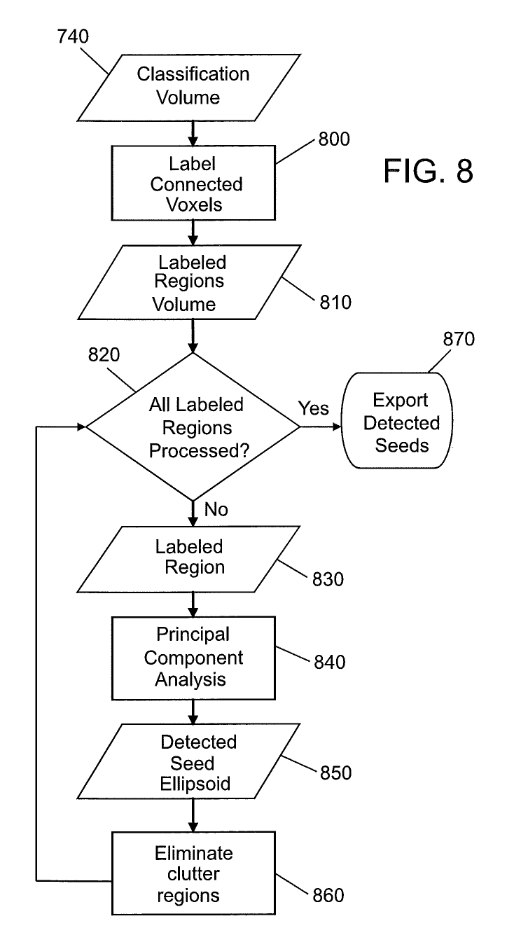

FIG. 7 shows a method of analyzing a reflectivity volume 700 according to one embodiment of the present invention. Ultimately, and as described in FIG. 8, the input reflectively volume 700 is analyzed to create “blobs” and blob analysis results in the identification and localization of connected regions. The shapes and sizes of the resulting regions are used to distinguish seeds from clutter.

At a block 710, the reflectivity volume 700 is converted into a reflective power volume. The reflective power volume is the norm of the reflectivity volume 700 over a sliding window. The window size is set to the seed size in RF samples with a 50% overlap between integration regions.

At a block 720 the reflective power volume is converted into a filtered volume using, for example, a 3×3 median filter.

At a block 730, the filtered volume is converted into a classification volume 740 by setting to all voxels below a threshold value to zero. Generally, the threshold value is chosen heuristically to make the best trade-off between spurious seed detections and missed seeds.

Refering now to FIG. 8, aspects of the process called “blob analysis” are illustrated. First, the classification volume 740 is converted into a labeled regions volume 810. At a block 800, connected clusters of cells having a non-zero value are labeled. In an example of labeling, the classification volume 740 is scanned, and preliminary labels are assigned to all non-zero voxels of the classification volume 740. The label equivalences are recorded in a union-find table. Equivalence classes are resolved using a union-find algorithm. Next, the voxels within the labeled regions volume 810 are re-labeled based on the resolved equivalence classes to produce a set of labeled regions. For each of the labeled regions (as determined at block 820), The process continues as illustrated in the flowchart of FIG. 8. Certain stages are repeated (according to the determination at block 820) for each labeled region 830.

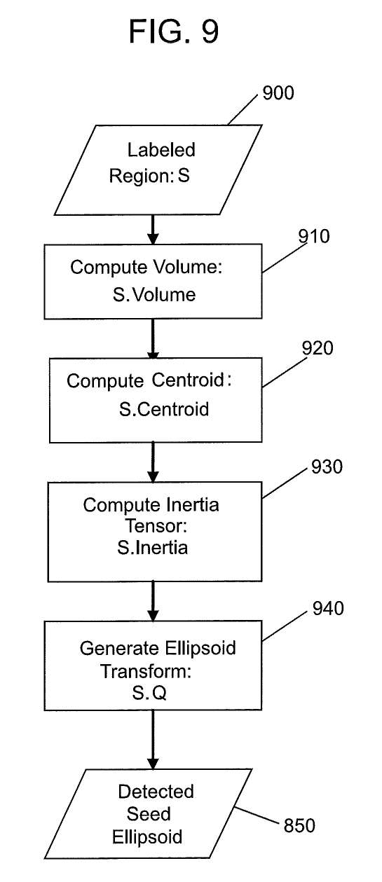

In a next stage, principal component analysis (PCA) 840 is performed. PCA is illustrated in the flowchart of FIG. 9. The steps of PCA 840 include computing the volume 910, centroid and covariance matrix. A homogenous transform matrix is computed which will transform the points of a unit sphere into an ellipsoid representing the detected seed. The centroid provides for determination of the seed position. The eigenvectors of the covariance matrix provide for determining the orientation of its ellipsoid axes, while axis lengths are determined by the covariance matrix eigenvectors. At a step 850 the seed ellipsoids are detected. In some embodiments, a heuristic is applied to distinguish valid seeds from clutter at a block 860.

In more detail, FIG. 9 shows the steps involved in PCA. From a labeled region 900 a volume (S.Volume) is computed at a block 910, a centroid (S.Centroid) is computed at a block 920 and an inertia tensor (S.Inertia) is computed at a block 930. At a block 940 an ellipsoid transform 940 is then generated at a block 940. The processing in each of these blocks is described in greater detail below.

At a block 920, as described above, the centroid S.Centroid is computed. S.Centroid may be computed where:

$$[ X , Y , Z ] = [ \frac{{\sum }_{i = 1}^{N} {x}_{i}}{N} , \frac{{\sum }_{i = 1}^{N} {y}_{i}}{N} , \frac{{\sum }_{i = 1}^{N} {z}_{i}}{N} ]$$

for N=S.Volume (the number of voxels contained by the region) and [xᵢ, yᵢ, zᵢ] is the coordinate of the ith voxel in the region.

At a block 930 the inertial tensor S.Inertia is computed. S.Inertia may be computed where:

$$Covariance matrix C [ 3 \times 3 ] = [ \begin{matrix}{C}_{xx} & {C}_{xy} & {C}_{xz} \\ {C}_{yx} & {C}_{yy} & {C}_{yz} \\ {C}_{zx} & {C}_{zy} & {C}_{zz}\end{matrix} ] where , \\ {C}_{xx} = \frac{{\sum }_{i = 1}^{N} {( {x}_{i} - \overline{X} )}^{2}}{N} , {C}_{xy} = \frac{{\sum }_{i = 1}^{N} ( {x}_{i} - \overline{X} ) ( {y}_{ki} - \overline{Y} )}{N} , \\ {C}_{xz} = \frac{{\sum }_{i = 1}^{N} ( {x}_{i} - \overline{X} ) ( {z}_{i} - \overline{Z} )}{N}$$

$${C}_{yx} = {C}_{xy} , {C}_{yy} = \frac{{\sum }_{i = 1}^{N} {( {y}_{i} - \overline{Y} )}^{2}}{N} , {C}_{yz} = \frac{{\sum }_{i = 1}^{N} ( {y}_{k} - \overline{Y} ) ( {z}_{i} - \overline{Z} )}{N}$$

$${C}_{zx} = {C}_{xz} , {C}_{zy} = {C}_{yz} , {C}_{zz} = \frac{{\sum }_{i = 1}^{N} {( {z}_{i} - \overline{Z} )}^{2}}{N}$$

For N=S.Volume and [ X̅, Y̅, Z̅]=S.Centroid and [xᵢ, yᵢ, zᵢ] is the coordinate of the ith voxel in the region.

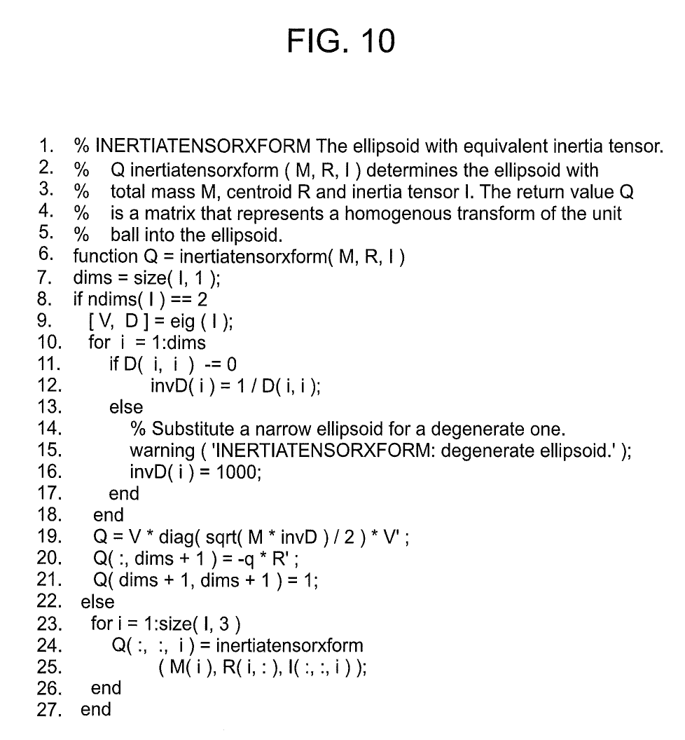

At a block 940 the ellipsoid transform S.Q is generated by computing the homogenous transform matrix Q from the unit ball to ellipsoid with Labeled Regions S.Inertia. This may include computing eigenvectors V and eigenvalues D from S.Inertia. The homogenous transform matrix Q may be computed using the Matlab source listing in FIG. 10.

In summary, application of computer vision techniques results in a collection of ellipsoids that correspond to hard objects detected in the imaged volume. The ellipsoids can be projected on to the plane of the ultrasound B-mode display for viewing by a clinician, or can be incorporated into software that performs dosimetry calculations. An example of this process applied to prostate brachytherapy is shown in FIG. 6.

Of course, embodiments of the present invention may be used in a variety of applications, such as localizing embedded markers within the breast for surgical procedures, assisting in intra-operative visualization of ablation procedures, and non-medical applications such as seismic data processing.

In support of the teachings herein, various analysis components may be used, including digital and/or an analog systems. The system may have components such as a processor, storage media, memory, input, output, communications link (wired, wireless, optical or other), user interfaces, software programs, signal processors (digital or analog) and other such components (such as resistors, capacitors, inductors and others) to provide for operation and analyses of the apparatus and methods disclosed herein in any of several manners well-appreciated in the art. It is considered that these teachings may be, but need not be, implemented in conjunction with a set of computer executable instructions stored on a computer readable medium, including memory (ROMs, RAMs), optical (CD-ROMs), or magnetic (disks, hard drives), or any other type that when executed causes a computer to implement the method of the present invention. These instructions may provide for equipment operation, control, data collection and analysis and other functions deemed relevant by a system designer, owner, user or other such personnel, in addition to the functions described in this disclosure.

As described above, embodiments can be embodied in the form of computer-implemented processes and apparatuses for practicing those processes. In exemplary embodiments, the invention is embodied in a computer program product including computer program code executed by one or more network elements. Embodiments include computer program code containing instructions embodied in tangible media, such as floppy diskettes, CD-ROMs, hard drives, or any other computer-readable storage medium, wherein, when the computer program code is loaded into and executed by a computer, the computer becomes an apparatus for practicing the invention. Embodiments include computer program code, for example, whether stored in a storage medium, loaded into and/or executed by a computer, or transmitted over some transmission medium, such as over electrical wiring or cabling, through fiber optics, or via electromagnetic radiation, wherein, when the computer program code is loaded into and executed by a computer, the computer becomes an apparatus for practicing the invention. When implemented on a general-purpose microprocessor, the computer program code segments configure the microprocessor to create specific logic circuits.

Further, various other components may be included and called upon for providing for aspects of the teachings herein. For example, an ultrasound system and various sub-components thereof, a power supply (e.g., at least one of a generator, a remote supply and a battery), a magnet, electromagnet, sensor, electrode, transmitter, receiver, transceiver, antenna, controller, optical unit, electrical unit or electromechanical unit may be included in support of the various aspects discussed herein or in support of other functions beyond this disclosure.

One skilled in the art will recognize that the various components or technologies may provide certain necessary or beneficial functionality or features. Accordingly, these functions and features as may be needed in support of the appended claims and variations thereof, are recognized as being inherently included as a part of the teachings herein and a part of the invention disclosed.

While the invention has been described with reference to exemplary embodiments, it will be understood that various changes may be made and equivalents may be substituted for elements thereof without departing from the scope of the invention. In addition, many modifications will be appreciated for adapting a particular instrument, situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims.

What is claimed is:

1. A method for providing an ultrasound image data, the method comprising:
assembling data from ultrasound image data of a subject;
performing linear wave inversion on the assembled data to provide a reflectivity image;
performing vision processing on the reflectivity image to identify and localize features within the ultrasound image; and
displaying the ultrasound image including the features.

2. The method of claim 1, wherein assembling includes:
amplifying electrical signals received by an ultrasound scanner; and
demodulating and digitizing the electrical signals to produce the assembled data.

3. The method of claim 1, wherein performing linear wave inversion includes:
creating a reflectivity basis.

4. The method of claim 3, wherein the reflectivity basis is a discrete reflectivity basis.

5. The method of claim 3, wherein the reflectivity basis is represented as a matrix including a plurality of basis elements, each basis element forming a column of the matrix.

6. The method of claim 5, wherein a pre-image of a first basis element overlaps with a pre-image of a second basis element.

7. The method of claim 1, wherein the assembled data represents an RF image and is expressed as a column vector.

8. The method of claim 7, wherein the reflectivity basis maps the reflectivity image vector to the RF image.

9. The method of claim 7, wherein the reflectivity image is expressed as a column vector.

10. The method of claim 8, wherein the reflectivity image is estimated using singular value decomposition.

11. The method of claim 1, wherein performing vision processing includes:
creating a classification volume from the reflectivity volume, the classification volume including blobs; and
performing blob analysis on the classified volume.

12. The method of claim 11, wherein creating the classification volume includes:
converting the reflectivity volume into a reflective power volume;
converting the reflective power volume into a filtered volume; and
setting all filtered values below a threshold to zero.

13. The method of claim 12, wherein converting the reflective power volume into a filtered volume includes:
applying a median filter.

14. The method of claim 11, wherein performing blob analysis includes:
labeling connected voxels in the classification volume to create labeled regions; and
performing principal component analysis of each labeled region.

15. The method of claim 14, wherein performing principal component analysis includes:
computing the volume of the labeled region;
determining a location of a centroid of the labeled region; and
computing the covariance matrix for the labeled region.

16. The method of claim 15, further comprising:
eliminating clutter regions.

17. The method of claim 2, wherein assembling further includes:
generating an RF volume by sampling ultrasound intensity at various points within the imaged volume.

18. The method of claim 17, wherein a pre-image of a first basis element overlaps with a pre-image of a second basis element.

19. A system for identifying hard objects, the system comprising:
an ultrasound scanner that creates an RF image representing an imaged area;
a signal processor that converts the RF image to a reflectivity image by performing linear wave inversion; and
a computer vision processor coupled to the signal processor to locate hard objects in the reflectivity image.

20. The system of claim 19, further comprising:
means for displaying the reflective image including the hard objects.

21. The system of claim 19, further comprising:
means for performing dosimetry calculations based on the reflectivity image and the location of the hard objects.

22. A method for determining locations of one or more element of interest in a subject, the method comprising:
assembling data from ultrasound image data of the subject;
performing linear wave inversion on the assembled data to provide a reflectivity image; and
analyzing the reflectivity image to determine the location of elements of interest.

23. The method of claim 22, wherein analyzing includes performing dosimetry analysis.

24. The method of claim 22, further comprising:
creating a matrix representing a reflectivity basis including a plurality of basis elements, each basis element forming a column of the matrix.

25. The method of claim 24, wherein the pre-image of a first basis element overlaps with the pre-image of a second basis element.

26. The method of claim 22, wherein the assembled data represents an RF image and is expressed as a column vector.

27. The method of claim 26, wherein a matrix representing a reflectivity basis maps the reflectivity image to the RF image.

28. A computer program product for determining locations of one or more element of interest in a subject, the computer program product comprising:
a storage medium readable by a processing circuit and storing instructions for execution by the processing circuit for facilitating a method including:
assembling data from ultrasound image data of the subject;
performing linear wave inversion on the assembled data to provide a reflectivity image; and
analyzing the reflectivity image to determine the location of elements of interest.
